(19) United States
(12) Patent Application Publication
(10) Pub. No.: US 2017/0095307 A1
(43) Pub. Date: Apr. 6, 2017

(54) Surgical Drape Cinch

(71) Applicant: Matthew Minwu Kang, Saint Paul, MN (US)

(72) Inventor: Matthew Minwu Kang, Saint Paul, MN (US)

(21) Appl. No.: 15/277,831

(22) Filed: Sep. 27, 2016

Related U.S. Application Data
(60) Provisional application No. 62/235,933, filed on Oct. 1, 2015.

(51) Int. Cl. A61B 46/20

(52) U.S. Cl. CPC A61B 46/20

(57) ABSTRACT

A surgical covering cinch that improves visibility during a surgery. The cinch makes use of a drape, a pair of fasteners, a cable system, and a surgical access hole. The drape is used to cover the body of a patient. The surgical access hole passes through the center of the drape. The pair of fasteners are mounted onto opposite edges of the drape. The cable system is connected to the fasteners and enables a user to move them as desired. By attaching the pair of fasteners to the drape and tensioning the cable system, a user is able to cinch the drape around the body of a patient to improve visualization and patient temperature regulation.

The current application claims a priority to the U.S. Provisional Patent application Ser. No. 62/235,933 filed on Oct. 1, 2015.

### FIELD OF THE INVENTION

The present invention relates generally to a surgical drape cinching device. More specifically, the present invention is a unique surgical drape cinching device designed to allow maximum visualization around the draped object, allow easier passage of medical equipment past the drapes, and increase patient body temperature more efficiently.

### BACKGROUND OF THE INVENTION

Traditional surgical drapes are designed to reduce the distractions and contaminants in the area of a patient's body that is being operated on. These devices are used for any number of surgeries and provide a surgeon with an unhindered and clean area in which to perform surgery. Traditional surgical drapes can hang deep below the operating table and hinder visualization of floor tools, floor pedals, and hinder access for medical equipment. In addition, an un-cinched drape can be a source of body temperature loss for the patient having surgery.

It is an objective of the present invention to provide a unique cinching mechanism that enables a surgeon to connect the edges of a surgical drape to each other. This is accomplished by mounting detachable fasteners to two opposite ends of the surgical drape. The detachable fasteners are connected or drawn to each other below the surgical table. As a result, the surgical drape is able to cinch around the body of a patient and trap in heat, improve visualization, and organize tubes/lines/cables.

### BRIEF DESCRIPTION OF THE DRAWINGS

FIG. 1 is a top view of the present invention.

FIG. 2 is a top view of the present invention in the cinched configuration.

FIG. 3 is a bottom view of the present invention in the cinched configuration.

FIG. 4 is a left-side view of the present invention in the cinched configuration; dashed lines indicate the body of a patient and an operating table.

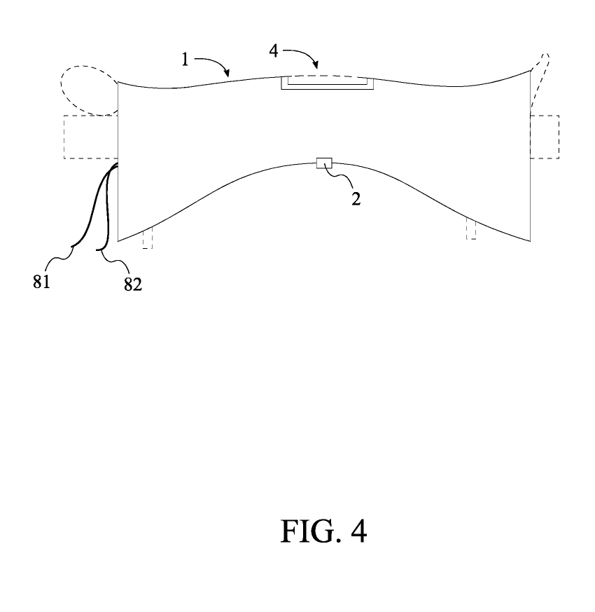

FIG. 5 is a top exploded view of the present invention.

FIG. 6 is a bottom exploded view of the present invention.

FIG. 7 is a top view of one embodiment of the present invention.

### DETAIL DESCRIPTIONS OF THE INVENTION

All illustrations of the drawings are for the purpose of describing selected versions of the present invention and are not intended to limit the scope of the present invention.

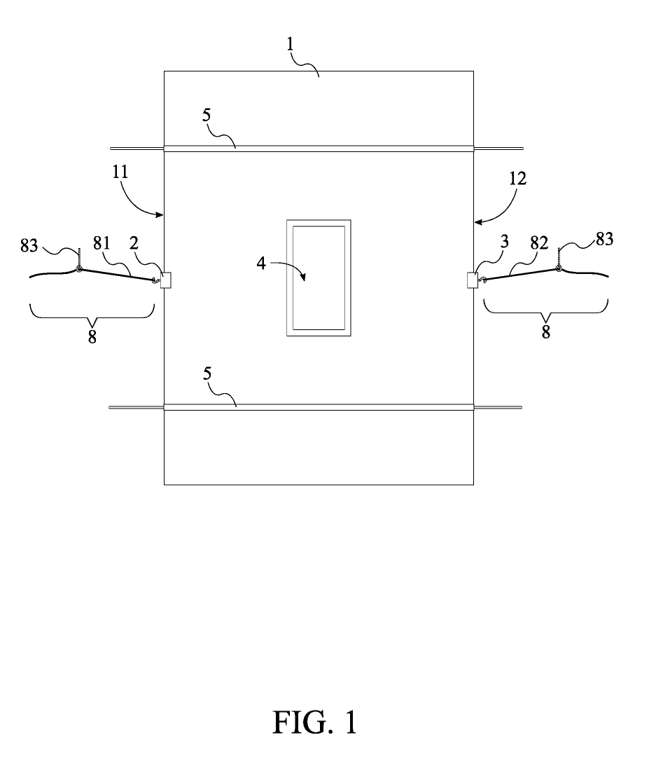

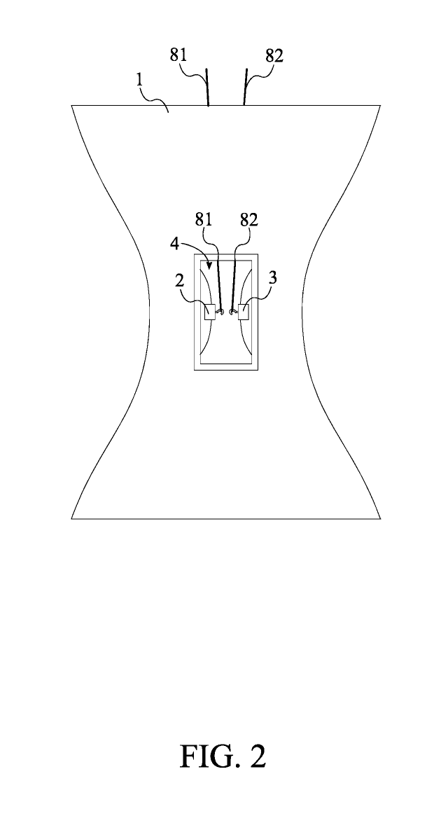

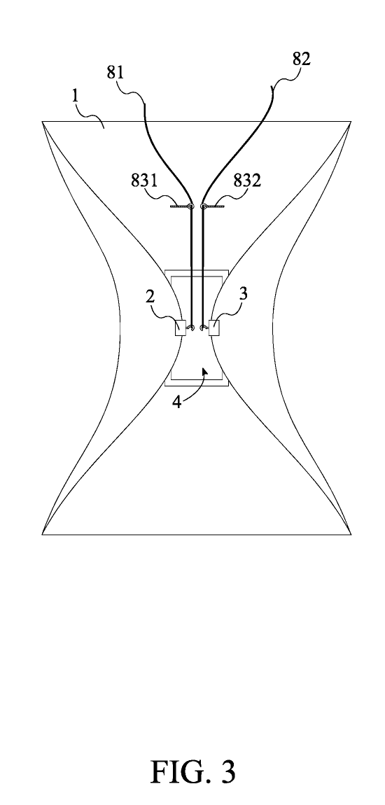

In reference to FIG. 1, FIG. 2, and FIG. 3, the preferred embodiment of the present invention is a cinch for enhancing visibility during surgery. The present invention is a device that enables a surgeon to perform surgeries without being hampered by poor visualization of floor equipment and trailing surgical drapes or instrument tubes. The present invention is a device that improves the ability to maintain an optimal patient body temperature by improving insulation. The present invention comprises a drape 1, a first fastener 2, a second fastener 3, a fastener adjustment assembly 8, and a surgical access hole 4. The drape 1 is a flexible piece of fabric that can be laid over the body of a patient undergoing surgery. The drape 1 comprises a first edge 11 and a second edge 12. The first edge 11 and the second edge 12 are reinforced edges of the drape 1 that enable external devices, such as fasteners, to be attached to the drape 1. The fastener adjustment assembly 8 is an adjustable cable system that is employed by users to pull the first fastener 2 and the second fastener 2 into an orientation that enhances visualization. The fastener adjustment assembly 8 comprises a first tether 81 and a second tether 82. The first edge 11 and the second edge 12 are positioned opposite to each other across the drape 1. As a result, the external fasteners can be attached to the drape 1 at positions which facilitate cinching the drape 1 around the body of a patient. The surgical access hole 4 traverses through the drape 1. The surgical access hole 4 is positioned equidistant in between the first edge 11 and the second edge 12 so that the body of a patient can be accessed through the surgical access hole 4 while the first edge 11 and the second edge 12 are touching.

In reference to FIG. 1, FIG. 2, and FIG. 3, in the present invention the first fastener 2 is mounted onto the drape 1 adjacent to the first edge 11. As a result, the first fastener 2 can be used to maintain the drape 1 in a cinched configuration. The second fastener 3 is mounted onto the drape 1 adjacent to the second edge 12. Consequently, the second fastener 3 can be attached to the first fastener 2 to maintain the drape 1 in a cinched configuration. The first fastener 2, the surgical access hole 4, and the second fastener 3 are positioned collinear to each other, so that the drape 1 is able to wrap around the body of a patient. The first tether 81 is connected to the first fastener 2, opposite to the first edge 11. Similarly, the second tether 82 is connected to the second fastener 3, opposite to the second edge. Consequently, as tension is placed on the first tether 81 and the second tether 82, the first fastener 2 and the second fastener 3 are brought together; effectively cinching the first edge 11 and the second edge 12 of the drape 1. This enables a surgeon to more consistently visualize and access any foot pedals that are located beneath the patient's surgical table. Additionally, this enables the drape 1 to function as a sleeve for collecting trailing cables and tubes. To accomplish this, the drape ledge 11 is held adjacent to the second edge 12 As a result, the drape 1 is able to form a tube that wraps around the body of a patient, as well as the surgical table on which the patient is resting. The first tether 81 and the second tether 82 are preferably ropes.

In reference to FIG. 1 and FIG. 2, the present invention further comprises an at least one adjustable cinch 5. The at least one adjustable cinch 5 is an adjustable strapping device used to tighten the drape 1 around the body of a patient, while the drape 1 is in the cinched configuration. The at least one adjustable cinch 5 is positioned offset from the surgical access hole 4 so that the drape 1 can be tightened around the body of a patient without impeding a surgeon's ability to access the patient's body through the surgical access hole 4. The at least one adjustable cinch 5 traverses from adjacent to the first edge 11, across the drape 1, and adjacent to the second edge 12. Consequently, the at least one adjustable cinch 5 facilitates securing the drape 1 in the cinched configuration.

In reference to FIG. 1, FIG. 2, and FIG. 3, in the present invention the fastener adjustment assembly 8 further comprises at least one eye bolt 83. The at least one eye bolt 83 is shaped similarly to a traditional eye bolt. The first tether 81 and the second tether 82 traverse through the at least one eyebolt 83, so that a user is able to pull the first edge 11 towards the second edge 12. Thus moving the drape 1 into the cinched configuration. The at least one eye bolt 83 is preferably attaches to a surgical table. In some embodiments, the at least one eye bolt 83 further comprises a first eye bolt 831 and a second eye bolt 832. The first eye bolt 831 and the second eyebolt 832 are attached to a surgical table, offset from each other. The first tether 81 traverses through the first eye bolt 831 so that the first tether 81 can be pulled in a direction independent of the second tether 82. Similarly, the second tether 82 traverses through the second eye bolt 832 so that the second tether 82 can be pulled in a direction independent of the first tether 81

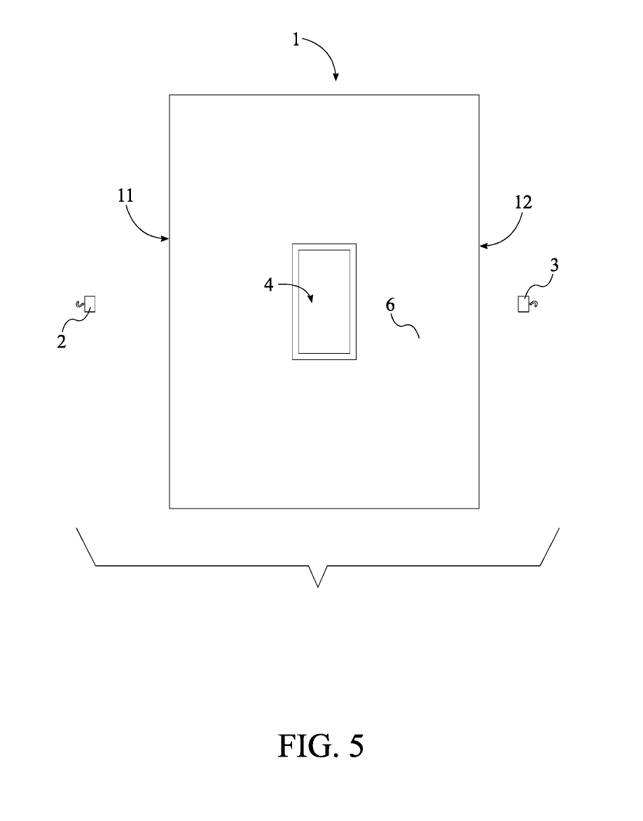

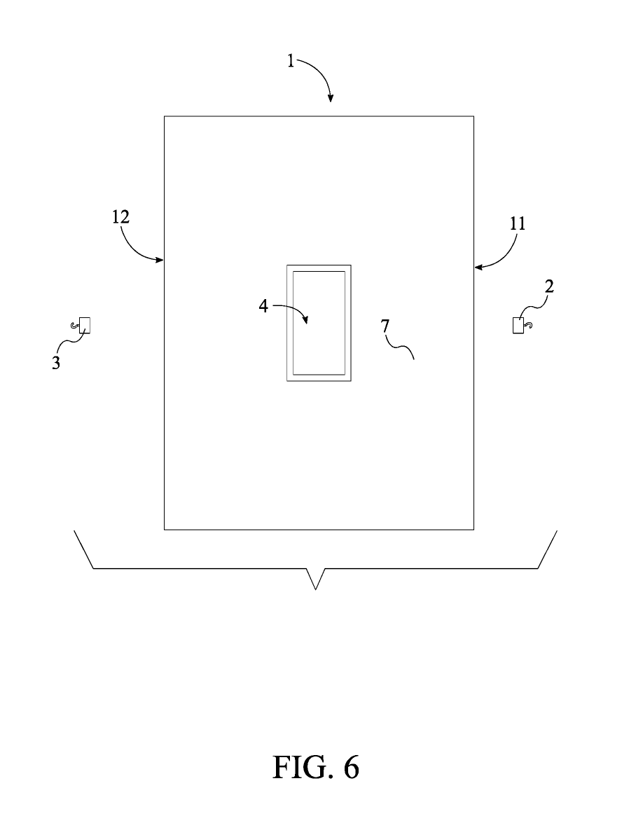

In reference to FIG. 5 and FIG. 6, the present invention further comprises a first face 6 and a second face 7. The first face 6 is a surface that is adjacently connected to the drape 1. Similarly, the second face 7 is adjacently connected to the drape 1, opposite to the first face 6. As a result, the user is able to clearly differentiate the first face 6 of the drape 1 from the second face 7 of the drape 1. A first alternative embodiment of the first fastener 2 comprises a length of the first fastener 21 along the length of the first edge 111 is less than the length of the first edge 111. Similarly, a first alternative embodiment of the second fastener 3 comprises a length of the second fastener 31 along the length of the second edge 121 is less than the length of the second edge 121. The length of the first fastener 21 and the length of the second fastener 31 are equal to each other. Consequently, the first fastener 2 is able to attach to a correspondingly sized second fastener 3.

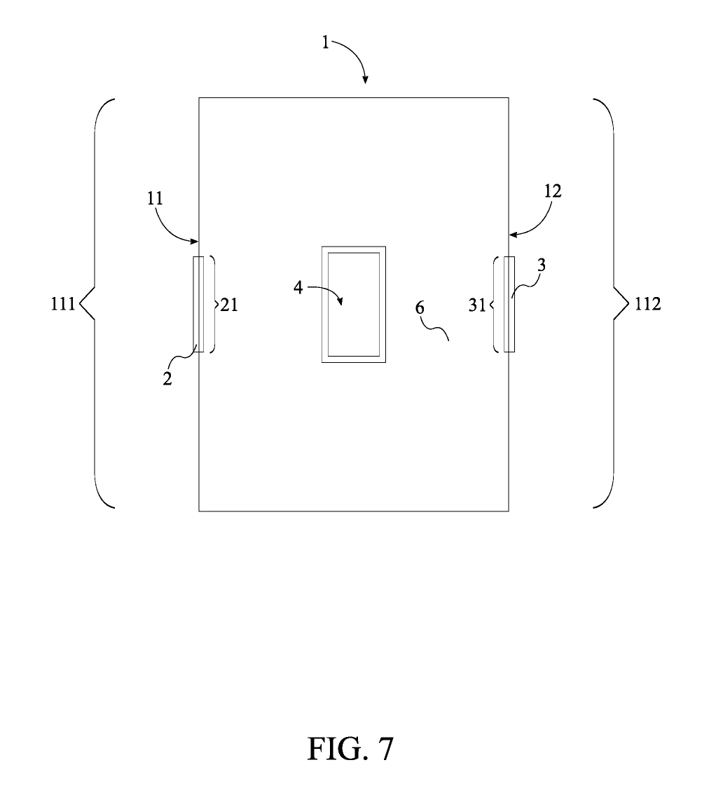

In reference to FIG. 7, a second alternative embodiment of the first fastener 2 comprises a length of the first fastener 21 along the length of the first edge 111 is equal to the length of the first edge 111. Similarly, a second alternative embodiment of the second fastener 3 comprises a length of the second fastener 31 along the length of the second edge 121 is equal to the length of the second edge 121. The length of the first fastener 21 and the length of the second fastener 31 are equal to each other. Consequently, the first fastener 2 is able to attach to a correspondingly sized second fastener 3.

In reference to FIG. 1 and FIG. 7, in a first alternative embodiment of the present invention, the first fastener 2 is a plurality of first fasteners 2. Similarly, in the first alternative embodiment of the present invention, the second fastener 3 is a plurality of second fasteners 3. As a result, each of the first fasteners 2 contained in the plurality of first fasteners 2 is able to attach to a corresponding second fastener 3 in the plurality of second fasteners 3. In a second alternative embodiment of the present invention, the first fastener 2 and the second fastener 3 are detachably mounted onto the drape 1. In a second alternative embodiment of the first fastener 2 and the second fastener 3, the first fastener 2 and the second fastener 3 are hooks. In a third alternative embodiment of the first fastener 2 and the second fastener 3, the first fastener 2 and the second fastener 3 are interlocking clasps. In a fourth alternative embodiment of the first fastener 2 and the second fastener 3, the first fastener 2 and the second fastener 3 are corresponding hook-and-loop pads. In a fifth alternative embodiment of the first fastener 2 and the second fastener 3, the first fastener 2 and the second fastener 3 are adhesive pads. In a sixth alternative embodiment of the first fastener 2 and the second fastener 3, the first fastener 2 and the second fastener 3 are magnets. In a sixth alternative embodiment of the first fastener 2 and the second fastener 3, the first fastener 2 and the second fastener 3 are clamps.

Although the invention has been explained in relation to its preferred embodiment, it is to be understood that many other possible modifications and variations can be made without departing from the spirit and scope of the invention as hereinafter claimed.

What is claimed is:

1. A surgical drape cinch for enhancing visibility during surgery comprises:
a drape;
a first fastener;
a second fastener;
fastener adjustment assembly;
a surgical access hole;
the drape comprises a first edge and a second edge;
the fastener adjustment assembly comprises a first tether and a second tether;
the first edge and the second edge being positioned opposite to each other across the drape;
the surgical access hole traversing through the drape;
the surgical access hole being positioned equidistant in between the first edge and the second edge;
the first fastener being mounted onto the drape, adjacent to the first edge;
the second fastener being mounted onto the drape, adjacent to the second edge;
the first fastener, the surgical access hole, and the second fastener being positioned collinear to each other;
the first tether being connected to the first fastener, opposite to the first edge;
the second tether being connected to the second fastener, opposite to the second edge

2. The surgical drape cinch for enhancing visibility during surgery as claimed in claim 1 comprises:
wherein the drape, the first fastener, and the second fastener are arranged into a cinched configuration;
the first edge being held adjacent to the second edge;

3. The surgical drape cinch for enhancing visibility during surgery as claimed in claim 1 comprises:
an adjustable cinch;
the adjustable cinch being positioned offset from the surgical access hole;
the adjustable cinch traversing from adjacent to the first edge, across the drape, and to adjacent to the second edge;

4. The surgical drape cinch for enhancing visibility during surgery as claimed in claim 4 wherein the first tether and the second tether are ropes;

5. The surgical drape cinch for enhancing visibility during surgery as claimed in claim 1 comprises:
the fastener adjustment assembly further comprises at least one eye bolt;
the first tether and the second tether traversing through the at least one eye bolt;

6. The surgical drape cinch for enhancing visibility during surgery as claimed in claim 5 wherein the at least one eye bolt is attached to a surgical table;

7. The surgical drape cinch for enhancing visibility during surgery as claimed in claim 5 comprises:
the at least one eye bolt comprises a first eye bolt and a second eye bolt;
the first eyebolt and the second eyebolt being attached to a surgical table offset from each other;
the first tether traversing through the first eye bolt;
the second tether traversing through the second eye bolt;

8. The surgical drape cinch for enhancing visibility during surgery as claimed in claim 1 comprises:
a length of the first fastener along a length of the first edge being less than the length of the first edge;
a length of the second fastener along a length of the second edge being less than the length of the second edge;
the length of the first fastener and the length of the second fastener being equal to each other;

9. The surgical drape cinch for enhancing visibility during surgery as claimed in claim 1 comprises:
a length of the first fastener along a length of the first edge being equal to the length of the first edge;
a length of the second fastener along a length of the second edge being equal to the length of the second edge;
the length of the first fastener and the length of the second fastener being equal to each other

10. The surgical drape cinch for enhancing visibility during surgery as claimed in claim 1 comprises:
a first face;
a second face;
the first face being adjacent connected to the drape body;
the second face being adjacently connected to the drape body, opposite to the first face;

11. The surgical drape cinch for enhancing visibility during surgery as claimed in claim 1 wherein the first fastener and the second fastener are hooks.

12. The surgical drape cinch for enhancing visibility during surgery as claimed in claim 1 wherein the first fastener and the second fastener are interlocking clasps.

13. The surgical drape cinch for enhancing visibility during surgery as claimed in claim 1 wherein the first fastener and the second fastener are corresponding hook-and-loop pads.

14. The surgical drape cinch for enhancing visibility during surgery as claimed in claim 1 wherein the first fastener and the second fastener are adhesive pads.

15. The surgical drape cinch for enhancing visibility during surgery as claimed in claim 1 wherein the first fastener and the second fastener are magnets

16. The surgical drape cinch for enhancing visibility during surgery as claimed in claim 1 wherein the first fastener and the second fastener are clamps.
